(19) United States
(12) Plant Patent Application Publication
(10) Pub. No.: US 2005/0120445 P1
(43) Pub. Date: Jun. 2, 2005

(54) HYBRID TEA ROSE PLANT NAMED 'HADDEL'

(50) ROSA HYBRIDA; HADDEL

(76) Inventor: Harvey Daniel Davidson, Orinda, CA (US)

Correspondence Address: HARVEY D. DAVIDSON, 3 EL VERANO RD., ORINDA, CA, 94563-1911, US

(21) Appl. No.: 10/724,476

(22) Filed: Dec. 1, 2003

(51) Int. Cl. A01H 5/00

(52) U.S. Cl. PLT/132

(57) ABSTRACT

A new and distinct rose cultivar of the Hybrid Tea class characterized by: having stems without prickles; large peach shaded flowers; flowers borne one to the stem; rapid repeat bloom; on upright bushy plants. The generic name of this plant is designated as ‘HADDEL’. It is being mmercial name of ‘SMOOTH DELIGHT’.

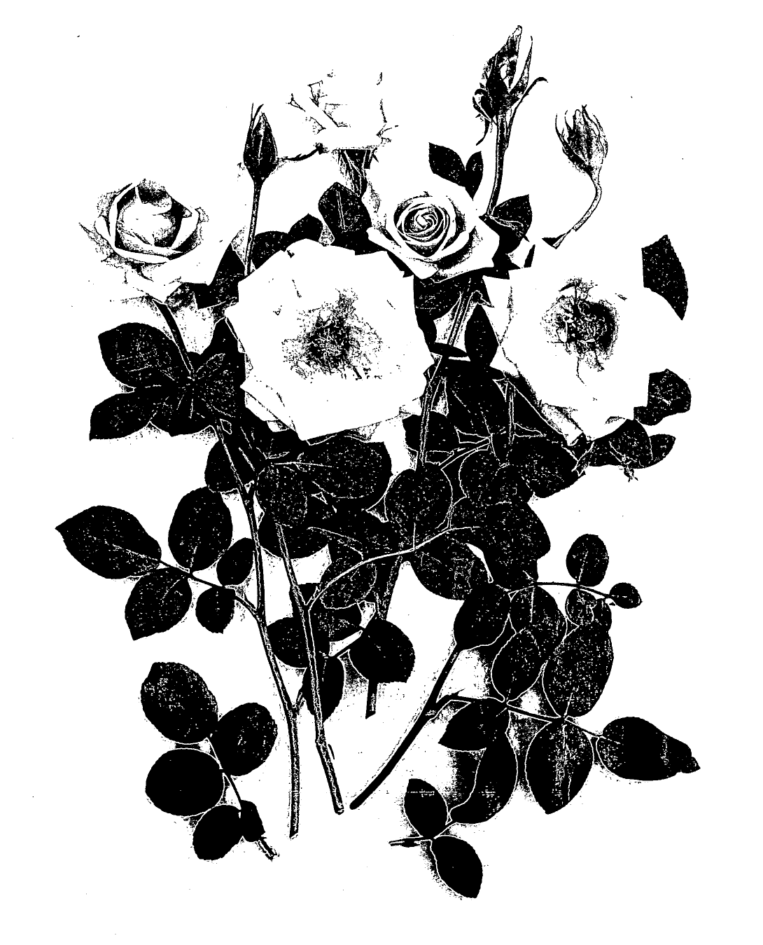

### BOTANICAL/COMMERCIAL CLASSIFICATION

Rosa hybrida/Hybrid Tea Rose Plant

The present invention relates to a new and distinctive cultivar of rose plant of the Hybrid Tea class, which was originated by my crossing an unnamed yellow seedling as the seed parent with ‘Smooth Sailing’ as the pollen parent.

Among the novel characteristics possessed by the new cultivar which distinguish it from:

(a) All other cultivars of which I am aware are:
(1) Prickles being absent from any stems.
(2) Large peach shaded flower.
(3) Single flower to the stem.
(4) Very consistent free bloomer.
(5) Upright bushy plant.

(b) Seed parent: Unnamed yellow hybrid tea seedling; few prickles.

Pollen parent: Tall growing; cream flowers; no prickles. Asexual reproduction of the new rose cultivar by budding onto the rootstock ‘Dr. Huey’, as performed at Wasco. Calif., shows that the aforementioned and all other distinctive characteristics come true to form and are established and transmitted through succeeding propagations.

The accompanying drawing shows typical specimens of the vegetative growth and flowers of the new cultivar in different stages of development and as depicted in color as nearly true as it is reasonably possible to make the same in a color illustration of this character.

The following is a detailed description of my new cultivar, with color terminology in accordance with The Royal Horticultural Society Colour Chart (RHSCC). The terminology used in the color description herein refers to plate numbers such as: ‘Yellow Group 16A’ found in Fan 1 of The Royal Horticultural Society Colour Chart.
Parentage:
   Seed parent.—Unnamed yellow seedling, hybrid tea.
   Pollen parent: ‘Smooth Sailing’, hybrid tea. Registered (IRAR). No USA patent.

The following observations are made of specimens grown outdoors in Orinda, Contra Costa County, Calif., in the month of October, 2003.

### FLOWER

Blooming habit:
A. Bud:
   (1) Size.—Sepals enclosed, 28-32 mm from base of sepals to top of bud.
   (2) Form.—Ovoid.
   (3) Sepals.—34-40 mm in length; 6 to 8 m extension beyond bud; no serration shown on sepal edges. Generally 3 out of 5 sepals show appendages on each side, 2 to 5 mm length. Sepals show light glandular development on edges amd outer surface. Heavy cotton matting on inner surface, fine matting on outer surface. Outside color: near to Yellow Green Grp. 146B with shadings of Greyed Orange Grp. 176A; Inside: Greyed Green Grp. 195A.
   (4) Peduncle.—Length — 6% to 7 cm. Surface: smooth; a few glandular hairs; no prickles. Strength: firm. Color: near Yel. Green 146D; with shades of Greyed Orange Group 176D.
   (5) Receptacle.—Shape: Oblong, 13-16 mm diameter; Color: same peduncle.
   (6) Color.—When sepals first divide, outside bud petals with shadings of Red Group 47B, C, D and shades near to Orange Grp. 23D.
B. Bloom:
   (1) Size.—When open: 11-12 cm.
   (2) Borne.—one flower per stem.
   (3) Form.—Globular.
   (4) Petalage.—Average — 34 to 36.
   (5) Color.—Early open: outside petals near to Red Group 56C; inner petals near to Orange Group 26C and D with shades of Yellow Orange Group 22C. Aging petals: Outer petals: near to Red Group 36C and D; inner petals: near to Yellow Orange 22C.
C. Petals:
   (1) Texture.—Firm.
   (2) Appearance.—Flexed.
   (3) Form.—Symmetrically spaced.
   (4) Arrangement.—Regular.
   (5) Petaloids in center.—Average 6 to 8.
   (6) Persistance.—Holds well; petals drop cleanly at end of bloom.
   (7) Fragrance.—Slight.
   (8) Lasting quality: On bush∴ 6-7 days; cut 4-6 days.

### REPRODUCTIVE ORGANS

A. Stamen:
   (1) Arrangement.—Regular around styles.
   (2) Number.—Average: 170-180.
   (3) Length.—Average: 5 to 10 mm from base.
   (4) Filaments.—Color: Close to Red Group 47C.
   (5) Anthers.—Color: Yel. Orange Grp. 21C.
   (6) Pollen: Color.—Same as (5).
B. Pistil:
   (1) Styles.—Length: 8-16 mm from center of ovary; number 130-140; color: near to Red Grp. 51B; sparsely coated with white hairs.
   (2) Stigma.—Color — Close to Yel. Grp. 12B.
   (3) Ovary.—Shape — oblong; 9-10 mm center cross section at sepal base.
C. Hips: Does not set seed.

### PLANT

A. Form: Upright.
B. Growth: Height: average 1.25 m; Width: average 0.75 m.
C. Stem length: Basal break to flower bud tip: average 1-1.25 m.
D. Foliage: Compound of 3 to 5 leaflets on bloom stem; 5 to 7 leaflets on main canes.
   (1) Quantity.—Stems well covered.
   (2) Color.—New foliage: Upper side near to Greyed Orange 177B; under side near Greyed Orange 177C; areas of Yel. Green 147B. Old foliage: upper side near to Yel. Green 147A; under side near to Yel. Green 147B.
   (3) Shape.—Mature 5 leaflet: Length: stipule base to tip of leaflet—11-13 cm. Center end leaflet: Oval and pointed. Length: 5-6 cm; Width: 4-4½ cm.
   (4) Texture.—Firm, leathery, gloss finish.
   (5) Edge.—Serration on outer edge: Average 4-5 points per 1 cm, with ½ to 1 mm peak.
   (6) Rachis.—Few glandular hairs on lower and upper side. No prickles on lower side.
   (7) Stipules.—Length 18-20 mm; auricle 4-5 mm. Edge of stipule to tip of auricle with many glandular hairs.
E. Resistance to disease: Powdery mildew, rust and blackspot: Good.
F. Wood:
   New wood.—Color close to Yel. Green 146C; shadings near to Greyed Orange Group 176D; Bark — smooth.
   Old wood.—Color close to Yel. Green 147B; Bark—smooth.
G. Prickles: None
H. Winter hardiness: Average.

1. A new and distinct rose cultivar of the Hybrid Tea class with essential features as follows:
1. Prickles are absent from any stems.
2. Large peach shaded flowers.
3. Flowers borne one to the stem.
4. Consistent free bloom.
6. Upright bushy plants of medium height.
substantially as shown herein and described.
